(19) United States
(12) Patent Application Publication
(10) Pub. No.: US 2026/0083688 A1
(43) Pub. Date: Mar. 26, 2026

(54) N-Acetylcysteine Amide Tablets Inhibit Reduction in Vision in Patients with Usher Syndrome Associated Retinitis Pigmentosa

(71) Applicant: Nacuity Pharmaceuticals, Inc., Fort Worth, TX (US)

(72) Inventors: G. Michael Wall, Fort Worth, TX (US); Jami Kern, Fort Worth, TX (US)

(21) Appl. No.: 19/338,733

(22) Filed: Sep. 24, 2025

Related U.S. Application Data
(60) Provisional application No. 63/698,705, filed on Sep. 25, 2024.

(51) Int. Cl. A61K 31/16; A61P 27/02

(52) U.S. Cl. CPC A61K 31/16; A61P 27/02

(57) ABSTRACT

Provided herein are compositions and methods for treating patients with Usher syndrome associated retinitis pigmentosa (UARP) comprising identifying that the subject has UARP and administering to the animal or human an effective amount of an N-acetylcysteine amide (NACA) sufficient to protect vision and inhibit degradation of Ellipsoid Zone (EZ) area and retinal sensitivity. In one example, the NACA formulation is doses at 50, 100, 200, 201, 210, 225, 250, 275, 300, 350, 400, 450, 500, 600, 700, 750, 800, 900, or 1,000 mg/day.

### CROSS-REFERENCE TO RELATED APPLICATIONS

This application claims priority to U.S. Provisional Application Ser. No. 63/698,705, filed Sep. 25, 2024, the entire contents of which are incorporated herein by reference.

### STATEMENT OF FEDERALLY FUNDED RESEARCH

None.

### TECHNICAL FIELD

The present invention relates in general to the field of N-acetylcysteine amide [NACA, NPI-001 (Nacuity code)] treatment for protection of vision, measured by effects on retinal sensitivity and inner segment (IS) layer ellipsoid zone (EZ or EZ area), in patients with Usher syndrome associated retinitis pigmentosa (UARP).

### BACKGROUND

Without limiting the scope of the disclosure, its background is described in connection with treating disease involving oxidative stress in the eye of patients, specifically the retina, specifically, patients with Usher syndrome associated retinitis pigmentosa (UARP).

Usher syndrome (USH) causes combined vision loss from a type of retinitis pigmentosa (RP) and hearing loss from inner ear dysfunction (Birch et al., 2020; Duncan et al., 2020). USH is the most common form of deaf-blindness, with an estimated worldwide prevalence of 4.4 to 16.6 per 100,000 people. USH is classified into 4 types, designated USH1, USH2, USH3, and USH4 that are further categorized into at least 11 subtypes associated with autosomal recessive mutations in specific genes (Zaw et al., 2022). RP is a heterogeneous set of inherited retinopathies with many disease-causing genes, many known mutations, and highly varied clinical consequences causing vision loss (Diager et al., 2013).

UARP is distinguishable from RP. In a multicenter natural history study of Usher syndrome type 2 (USH2A, N=80 patients) versus autosomal recessive nonsyndromic retinitis pigmentosa (ARRP, N=47 patients) patients, USH2A patients (UARP) were found to have worse Best Corrected Visual Acuity (BCVA), electroretinogram (ERG), and full-field stimulus testing (FST) results than ARRP patients (Birch et al., 2020). Participants with USH2 (UARP) had worse visual field sensitivity (VTOT and V30) than participants with ARRP, even after accounting for disease duration and age at enrollment (Duncan et al., 2020). A previous study comparing participants with USH2 (UARP) with ARRP due to biallelic USH2A sequence variants found that those with USH2 (UARP) had more severe visual impairment measured by visual field and visual acuity, occurring at least a decade earlier than those with ARRP (Pierrache et al., 2016). Similarly, in another study, ERG 30-Hz flicker amplitudes were lower in participants with USH2 (UARP) compared to ARRP (Sengillo et al., 2017). More severe truncating sequence variants have been reported in participants with USH2 (UARP) than ARRP, and hearing loss is also more severe in those with truncating USH2A (UARP) sequence variants compared to missense sequence variants (Hartell et al., 2016). Also, UARP is variable, e.g., USH3 patients showed significantly poorer visual field function than the USH2A patients (Plantinga et al., 2005). Therefore, it is not clear that a treatment for RP would also be more or less effective for UARP, versus different types of USH, or vice versa.

Differences in severity are attributed to the genetic diversity of inherited retinal diseases (IRDs). A retrospective epidemiological study (Karali et al., 2022) was conducted to determine the genetic basis of IRDs in a large Italian cohort (n=2790) followed at a single referral center. Mainly by next generation sequencing, potentially conclusive molecular diagnosis for 2036 patients (from 1683 unrelated families) were ascertained. A total of 1319 causative sequence variations in 132 genes, including 353 novel variants, and 866 possibly actionable genotypes for therapeutic approaches were identified. ABCA4 was the most frequently mutated gene (n=535; 26.3% of solved cases), followed by USH2A (n=228; 11.2%) and RPGR (n=102; 5.01%). The other 129 genes had a lower contribution to IRD pathogenesis (e.g. CHM 3.5%, RHO 3.5%; MYO7A 3.4%; CRB1 2.7%; RPE65 2%, RP1 1.8%; GUCY2D 1.7%). Seventy-eight genes were mutated in five patients or less. Mitochondrial DNA variants were responsible for 2.1% of cases.

The retina is a thin layer of tissue lining the back of the eye composed of light-sensing photoreceptor cells (i.e., rods and cones), which are responsible for converting light into electrical signals that the brain interprets as vision. Retinitis pigmentosa (RP) is a group of diseases in which the inciting event is a mutation that leads to the death of rod photoreceptors followed by death of cone photoreceptors. Accumulating experimental evidence has shown that oxidative stress is a pathogenic factor in RP. Antioxidants have been shown to improve biomarkers of oxidative stress and partially preserve cone function in the rdl mouse model of RP (Komeima et al., 2006; Komeima et al., 2007). It has been reported that biomarkers of oxidative stress in the aqueous humor are modified in patients with RP, protein carbonyl levels are increased, and the ratio of reduced glutathione to oxidized glutathione (GSH:GSSG) is decreased (U.S. Pat. No. 11,268,964).

Currently, there is no approved drug therapy that stops or slows the evolution of UARP or restores vision. The therapeutic approach is largely restricted to slowing down the degenerative process by sunlight protection and vitamin A supplementation, treating complications (cataract and macular edema), and helping patients to cope with the social and psychological impact of blindness. Although the Argis II Retinal Prosthesis System was approved by FDA in 2013 as an implanted device to treat adults with severe RP, it only produces the sensation of light, thereby helping patients identify the location or movement of objects and people; the device is not disease-modifying nor specific to UARP. In 2017, FDA approved LUXTURNA®, a gene therapy for the treatment of patients with confirmed biallelic RPE65 mutation-associated retinal dystrophy that leads to vision loss and may cause complete blindness in certain patients: this population comprises only about 1% of RP patients and does not include UARP patients.

The slow rate of progression in measures of visual function has been a deterrent for treatment trials in patients with UARP. Visual acuity typically remains stable until late in the disease's progression. Kinetic visual fields begin to constrict after a critical age, but the annual rate of decline varies from 5% to 20% depending on the isopter and RP genotype. Static perimetry, which evolved primarily for the detection of glaucomatous field defects, has been used as a primary outcome measure in clinical trials in RP as has the full-field ERG. However, trial durations of several years are necessary because the rate of progression in these measures is low, especially when compared with the intervisit variability (Birch et al., 2013).

Microperimetry, or fundus-tracked perimetry, is a precise static-automated perimetric technique to assess central retinal function (Buckley et al., 2021). The Macular Integrity Assessment (MAIA) microperimeter (MAIA, CenterVue Inc.) uses an LED light stimulus and a scanning laser to perform retinal imaging to determine sensitivity in the central retina.

Spectral-domain optical coherence tomography (SD-OCT) measures of anatomical or structural changes in the outer segment (OS) layer have been proposed as possible outcome measures for clinical trials of RP types. Ten laminar boundaries can be distinguished with current SD-OCT clinical devices, leading to 9 identifiable retinal layers. Of these, the most immediately relevant to progression in RP are the OS layer, the inner segment (IS) layer, and the outer nuclear layer. A previous study demonstrated that the region of the retina showing an IS ellipsoid zone (EZ or EZ area), previously referred to as the IS/OS border, can be directly related to the visual field boundary. SD-OCT measures of EZ area are typically obtained with a commercial device, i.e., Spectralis Heidelberg retina angiography+OCT (Heidelberg Engineering, Inc) (Birch et al., 2013).

As such, there still exists a need for novel compositions and methods for treatment of Usher syndrome associated retinitis pigmentosa (UARP).

### SUMMARY OF THE INVENTION

As embodied and broadly described herein, an aspect of the present disclosure relates to a method for treating an eye disease caused by oxidative damage in an animal or human in need thereof, the method comprising: administering to the animal or human an effective amount of an N-acetylcysteine amide (NACA) sufficient to protect vision and inhibit degradation of Ellipsoid Zone (EZ) area and retinal sensitivity. In one aspect, the eye disease is Usher syndrome associated retinitis pigmentosa (UARP). In another aspect, the NACA is provided orally, peritoneally, intravenously, dermally, bucally, sublingually, topically, topical ocularly, intraocularly, intravitreally, transmucosally, or by inhalation. In another aspect, the NACA inhibits the reduction in EZ area as measured by Spectral Domain Optical Coherence Tomography (SD-OCT). In another aspect, the NACA inhibits the reduction retinal sensitivity as measured by microperimetry. In another aspect, the NACA is dosed as one tablet per day or as multiple tablets per day. In another aspect, the NACA is dosed greater than 200 mg per day. In another aspect, the NACA is dosed at 50, 100, 200, 201, 210, 225, 250, 275, 300, 350, 400, 450, 500, 600, 700, 750, 800, 900, or 1,000 mg per day. In another aspect, the NACA is dosed for less than 6 months or for 6, 9, 12, 18, 24, or more than 24 months. In another aspect, the NACA dose does not reach an appreciable daily steady-state concentration.

As embodied and broadly described herein, an aspect of the present disclosure relates to a method for treating retinitis pigmentosa associated with Usher syndrome associated retinitis pigmentosa (UARP), the method comprising: identifying that the subject has Usher syndrome associated retinitis pigmentosa (UARP); and administering an effective amount of an N-acetylcysteine amide (NACA) sufficient to inhibit degradation of vision in Usher syndrome associated retinitis pigmentosa (UARP). In one aspect, the NACA inhibits degradation of Ellipsoid Zone (EZ) area and retinal sensitivity. In another aspect, the NACA inhibits a reduction in EZ area as measured by Spectral Domain Optical Coherence Tomography (SD-OCT). In another aspect, the NACA inhibits a reduction in retinal sensitivity as measured by microperimetry. In another aspect, the NACA is dosed as one tablet per day or as multiple tablets per day. In another aspect, the NACA is dosed greater than 200 mg per day. In another aspect, the NACA is dosed at 50, 100, 200, 201, 210, 225, 250, 275, 300, 350, 400, 450, 500, 600, 700, 750, 800, 900, or 1,000 mg per day. In another aspect, the NACA is dosed for less than 6 months or for 6, 9, 12, 18, 24, or more than 24 months. In another aspect, the NACA dose does not reach an appreciable daily steady-state concentration.

As embodied and broadly described herein, an aspect of the present disclosure relates to a method for treating an eye disease caused by oxidative damage in an animal or human in need thereof, the method comprising: identifying that the subject has retinitis pigmentosa associated with Usher syndrome (RP/USH); and administering an effective amount of an N-acetylcysteine amide (NACA) sufficient to inhibit loss of vision.

As embodied and broadly described herein, an aspect of the present disclosure relates to a method for the treatment of UARP in an animal or human that comprises administering to the animal or human a therapeutically effective amount of N-acetylcysteine amide (NACA or NPI-001). The drug product example in this patent is a tablet formulation of NACA for oral ingestion, but other formulations may be used. In one aspect, the NACA is provided in or with a pharmaceutically acceptable carrier. In another aspect, the NACA is administered intraocularly, subretinally, intravitreally, orally, intravenously, intramuscularly, topically, sublingually, or rectally. In another aspect, the NACA is administered in daily doses of about 2.87 to 7.4 mg/Kg. In another aspect, the NACA is administered in daily doses of about 1, 2, 3, 4, 5, 6, 7, 8, 9, 10, 15, 20, 25, 30, 35, 40, 45, to 50 mg/Kg. In another aspect, NACA is administered two or three times daily. In another aspect, NACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytouene (BHT), lecithin, propyl gallate, α-tocopherol, citric acid, ethylenediamine tetraacetic acid (EDTA), lipoic acid, sorbitol, tartaric acid, or phosphoric acid. In another aspect, the dose for administration is 200, 201, 250, 300, 333, 400, 500, 600, 700, 750, 800, 900, 1,000, 2,500, 5,000, 7,500, or 10,000 mg per dose. In another aspect, the dose for administration is 0.2-10, 0.201-0.34, 0.35-0.5, 0.5-1, 1-2, 1-3, 1-4, 1-5, 1-2.5, 2.5-3.5, 4-6, 5-8, 6-9, 7-10 grams per dose. In another aspect, the NACA is delivered orally via a mini-tablet, capsule, tablet, effervescent, dual release, mixed release, sachet, powder, or liquid. In another aspect, the NACA is administered prophylactically to prevent age-related macular degeneration. In another aspect, the animal is a human. In another aspect, the NACA is administered prophylactically to prevent reduction in retinal sensitivity or EZ area in patients with UARP. In another aspect, the NACA is administered prophylactically in adult patients to prevent reduction in retinal sensitivity or EZ area in patients with UARP. In another aspect, the NACA is administered prophylactically in pediatric patients to prevent reduction in retinal sensitivity or EZ area in patients with UARP. In another aspect, the NACA is administered prophylactically in adolescent patients to prevent reduction in retinal sensitivity or EZ area in patients with UARP.

As embodied and broadly described herein, an aspect of the present disclosure relates to a method for treating an eye disease caused by oxidative damage in an animal or human in need thereof, the method comprising: identifying that the subject has Usher syndrome associated retinitis pigmentosa (UARP); and providing an effective amount of an N-acetylcysteine amide (NACA) sufficient to inhibit degradation of vision. In one aspect, the eye disease is Usher syndrome associated retinitis pigmentosa (UARP). In another aspect, the NACA is provided orally, peritoneally, intravenously, dermally, bucally, sublingually, topically, topical ocularly, intraocularly, intravitreally, transmucosally, or by inhalation. In another aspect, the NACA inhibits the reduction in vision based on a primary endpoint. In another aspect, the NACA inhibits the reduction in vision based on a secondary endpoint. In another aspect, the NACA is dosed as multiple tablets per day. In another aspect, the NACA is dosed for at least 6 months, or 12 months. In another aspect, the NACA is dosed for 24 months. In another aspect, the NACA is dosed for more than 24 months. In another aspect, the NACA dose does not reach an appreciable daily steady-state concentration. In another aspect, the NACA is administered prophylactically to prevent reduction in retinal sensitivity or EZ area in patients with UARP. In another aspect, the NACA is administered prophylactically in adult patients to prevent reduction in retinal sensitivity or EZ area in patients with UARP. In another aspect, the NACA is administered prophylactically in pediatric patients to prevent reduction in retinal sensitivity or EZ area in patients with UARP. In another aspect, the NACA is administered prophylactically in adolescent patients to prevent reduction in retinal sensitivity or EZ area in patients with UARP.

As embodied and broadly described herein, an aspect of the present disclosure relates to a method for treating retinitis pigmentosa associated with Usher syndrome associated retinitis pigmentosa (UARP), the method comprising: identifying that the subject has Usher syndrome associated retinitis pigmentosa (UARP); and providing an effective amount of an N-acetylcysteine amide (NACA) sufficient to inhibit degradation of vision in Usher syndrome associated retinitis pigmentosa (UARP). In one aspect, the NACA inhibits the reduction in vision based on a primary endpoint. In another aspect, the NACA inhibits the reduction in vision based on a secondary endpoint. In another aspect, the NACA is dosed as multiple tablets per day. In another aspect, the NACA is dosed as two tablets, each containing 200, 201, 210, 225, 250, 275, 300, 350, 400, 450, 500, 600, 700, 800, 900, or 1,000 mg NACA, once daily. In another aspect, the NACA is dosed as two tablets, each containing 200, 201, 210, 225, 250, 275, 300, 350, 400, 450, 500, 600, 700, 800, 900, or 1,000 mg NACA, twice daily. In another aspect, the NACA is dosed as two tablets, each containing 200, 225, 250, 275, 300, 350, 400, 450, 500, 600, 700, 800, 900, or 1,000 mg NACA, thrice daily. In another aspect, the NACA Tablets are dosed for at least 6 months, or 12 months. In another aspect, the NACA Tablets are dosed for at least 24 months. In another aspect, the NACA is dosed for more than 24 months. In another aspect, the e NACA dose does not reach an appreciable daily steady-state concentration. In another aspect, the NACA is administered prophylactically to prevent reduction in retinal sensitivity or EZ area in patients with UARP. In another aspect, the NACA is administered prophylactically in adult patients to prevent reduction in retinal sensitivity or EZ area in patients with UARP. In another aspect, the NACA is administered prophylactically in pediatric patients to prevent reduction in retinal sensitivity or EZ area in patients with UARP. In another aspect, the NACA is administered prophylactically in adolescent patients to prevent reduction in retinal sensitivity or EZ area in patients with UARP.

### BRIEF DESCRIPTION OF THE DRAWINGS

For a more complete understanding of the features and advantages of the present invention, reference is now made to the detailed description of the invention along with the accompanying figure:

FIG. 1 shows statistically significant slowing of photoreceptor loss with N-acetylcysteine amide [NACA, NPI-001 (Nacuity code)] (solid blue line) treatment versus placebo (dashed red line) by mean change from baseline in EZ area by SD-OCT through the 24-month visit (NCT04355689).

FIG. 2 shows change from baseline retinal sensitivity [by microperimetry by Macular Integrity Assessment (MAIA)] over time, suggesting a treatment effect with N-acetylcysteine amide [NACA, NPI-001 (Nacuity code)] (solid blue line) versus placebo (dashed red line).

FIG. 3 shows that effects of N-acetylcysteine amide [NACA, NPI-001 (Nacuity code)] on EZ area (retinal structure) and retinal sensitivity (functional vision) are highly correlated (Pearson coefficient).

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

As described hereinabove, UARP is distinguishable from RP. USH2A patients (UARP) were found to have worse Best Corrected Visual Acuity (BCVA), electroretinogram (ERG), and full-field stimulus testing (FST) results than ARRP patients. Participants with USH2 (UARP) had worse visual field sensitivity (VTOT and V30) than participants with ARRP, even after accounting for disease duration and age at enrollment. Studies comparing participants with USH2 (UARP) with ARRP due to biallelic USH2A sequence variants found that those with USH2 (UARP) had more severe visual impairment measured by visual field and visual acuity, occurring at least a decade earlier than those with ARRP. In another study, ERG 30-Hz flicker amplitudes were lower in participants with USH2 (UARP) compared to ARRP. More severe truncating sequence variants have been reported in participants with USH2 (UARP) than ARRP, and hearing loss is also more severe in those with truncating USH2A (UARP) sequence variants compared to missense sequence variants. UARP is variable, e.g., USH3 patients showed significantly poorer visual field function than the USH2A patients. Therefore, it is not clear that a treatment for RP would also be more or less effective for UARP, versus different types of USH, or vice versa.

N-acetylcysteine amide (NACA or NPI-001) is being developed as a potential treatment for UARP with a randomized, placebo-controlled study ongoing (ClinicalTrials.gov Identifier: NCT04355689). The chemical structure of NPI-001:

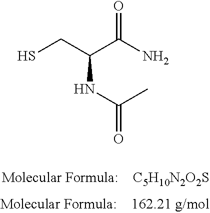

N-Acetylcysteine Amide (NACA). Orally administered N-Acetylcysteine amide (NACA, NPI-001) has been found to be a particularly effective antioxidant to treat retinitis pigmentosa (See U.S. patent application Ser. No. 15/523,665). The NACA can be dosed at 200, 225, 250, 275, 300, 350, 400, 450, 500, 600, 700, 800, 900, or 1,000 per day. the NACA is dosed as multiple tablets per day. The NACA can be dosed as two tablets, each containing 200, 225, 250, 275, 300, 350, 400, 450, 500, 600, 700, 800, 900, or 1,000 mg NACA, once daily. The NACA can be dosed as two tablets, each containing 200, 225, 250, 275, 300, 350, 400, 450, 500, 600, 700, 800, 900, or 1,000 mg NACA, twice daily. The NACA can be dosed as two tablets, each containing 200, 225, 250, 275, 300, 350, 400, 450, 500, 600, 700, 800, 900, or 1,000 mg NACA, thrice daily. The NACA can be dosed for at least 6 months, or 12 months. The NACA can be dosed for at least 24 months. The NACA can be dosed for more than 24 months. The NACA can be dosed such that the NACA does not reach an appreciable daily steady-state concentration.

As used herein, the terms “effective amount” or “effective doses” refer to that amount of an agent to product the intended pharmacological, therapeutic or preventive results. The pharmacologically effective amount results in the amelioration of one or more signs or symptoms of a disease or condition or the advancement of a disease or conditions, or causes the regression of the disease or condition. For example, a therapeutically effective amount preferably refers to the amount of a therapeutic agent that decreases vision loss, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such as treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as an improvement of symptoms, a cure, a reduction in disease signs or symptoms, extension of life, improvement in quality of life, or other effect generally recognized as positive by medical doctors familiar with treating the particular type of disease or condition.

As used herein, the term phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperitoneal, intraocular, intravitreal, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, the term “subject” refers to living organisms, in particular, humans. In certain embodiments, the living organism is an animal, in certain preferred embodiments, the subject is a mammal, in certain embodiments, the subject is a domesticated mammal or a primate including a non-human primate. Examples of subject include humans, monkeys, dogs, cats, mice, rates, cows, horses, goats, and sheep. A human subject may also be referred to as a subject or patient.

As used herein, the term “therapeutically effective amount,” refers to an amount of an agent which is effective, upon single or multiple does administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, PA, 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

The present invention is directed to the use of NACA to prevent and/or treat UARP. In one embodiment, the present invention includes a method for use of N-acetylcysteine amide (NACA) or (2R,2R′)-3,3′-disulfanediyl bis(2-acetamidopropanamide) (diNACA) for the prevention and/or treatment of UARP in a human that comprises administering to the human therapeutically effective amount of NACA. In some embodiments, the NACA is provided in or with a pharmaceutically acceptable carrier. In other embodiments, the NACA is administered intraocularly, subretinally, intravitreally, orally, intravenously, intramuscularly, topically, sublingually, or rectally.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the forgoing guidelines.

Ranges provided herein are understood to be shorthand for all of the values within the range.

As used herein, the embodiments of this invention are defined to include pharmaceutically acceptable derivatives thereof. A “pharmaceutically acceptable derivative” means any pharmaceutically salt, ester, salt of an ester, or other derivative of a compound of this invention which, upon administration to a recipient, is capable of providing (directly or indirectly) a compound of this invention. Particularly favored derivatives are those that increase the bioavailability of the compounds of this invention when such compounds are administered to a mammal (e.g., by allowing an orally administered compound to be more readily absorbed into the blood, to increase serum stability or decrease clearance rate of the compound) or which enhance delivery of the parent compound to a biological compartment (e.g., the brain or lymphatic system) relative to the parent species. Derivatives include derivatives where a group which enhances aqueous solubility or active transport through the gut membrane is appended to the structure of formulae described herein.

The embodiments of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-napthalenesulfonate, nicotinate, nitrate, palmoate, phosphate, picrate, pivalate, propionate, salicylate, succinate, sulfate, tartrate, tosylate, and undeconaoate. Salts derived from appropriate bases include alkali metal (e.g., sodium), alkaline earth metal (e.g., magnesium), ammonium and N-(alkyl)4+ salts. This invention also envisions the quaternization of any basic nitrogen-containing groups of the compounds disclosed herein. Water or oil-soluble or dispersible products may be obtained by such quaternization.

The embodiments of the invention can, for example, be administered by injection, intraocularly, intravitreally, subretinal, intravenously, intraarterially, subdermally, intramuscularly, or subcutaneously; or orally, buccally, nasally, transmucosally, directly to a diseased organ by catheter, topically, or in an ophthalmic preparation, with a dosage ranging from about 0.001 to about 100 mg/kg of body weight, or according to the requirements of the particular drug and more preferably from 0.5-10 mg/kg of body weight. It is understood that when a compound is delivered directly to the eye, considerations such as body weight have less bearing on the dose.

Frequency of dosing will depend on the agent administered, the progression of the disease or condition in the subject, and other considerations known to those of skill in the art. For example, pharmacokinetic and pharmacodynamics considerations for compositions delivered to the eye, or even compartments within the eye, are different, e.g., clearance in the subretinal space is very low. Therefore, dosing can be as infrequent as once a month, once every three months, once every six months, once a year, once every five years, or less. If systemic administration of antioxidants is to be performed in conjunction with administration of expression constructs to the subretinal space, it is expected that the dosing frequency of the antioxidant will be higher than the expression construct, e.g., one or more times daily, one or more times weekly.

Dosing may be determined in conjunction with monitoring of one or more signs or symptoms of the disease, e.g., visual acuity, visual field, night visions, etc. The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity ad course of the disease, condition or symptoms, the patient's disposition to the disease, condition or symptoms and the judgment of the treating physician.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPAN® and/or other similar emulsifying agents or bioavailability enhancers which are commonly used in the manufacture of pharmaceutically acceptable solid, liquid, or other dosage forms may also be used for the purposes of formulation.

In one or more embodiments, NACA or diNACA is administered in daily doses of about 0.5 to 150 mg/Kg. In other embodiments, NACA or diNACA is administered two or three times daily. In another aspect, NACA or diNACA is administered with a second active agent selected from ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

In some embodiments, the dose of NACA or diNACA for administration is 100, 150, 175, 200, 201, 300, 333, 400, 500, 600, 700, 750, 800, 900, 1,000, 2,500, 5,000, 7,500, or 10,000 mg per dose. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, the NACA or diNACA is delivered orally via a mini-tablet, capsule, tablet, effervescent, dual release, mixed release, sachet, powder, or liquid. In another aspect, the NACA is administered prophylactically to prevent and/or treat UARP.

### Example 1: Nacuity Pharmaceuticals, Inc., Clinical Trial C-18-04, ‘SLO RP’

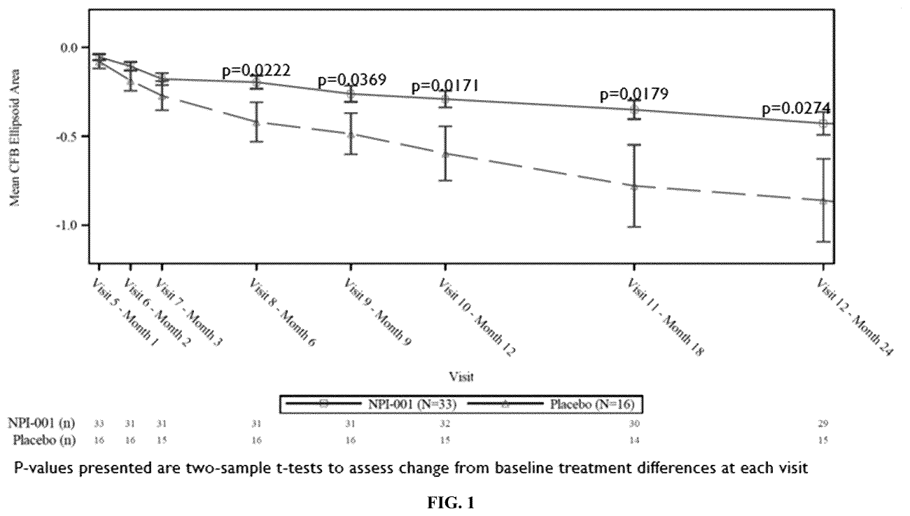

Nacuity Pharmaceuticals, Inc., completed Clinical Study C-18-04, i.e., “Safety and Efficacy of NPI-001 Tablets versus Placebo for Treatment of Retinitis Pigmentosa Associated with Usher Syndrome (SLO RP)” (ClinicalTrials.gov Identifier: NCT04355689). The SLO RP Trial, double-masked, placebo controlled and randomized (2 active (NPI-001 tablets at 500 mg/day): 1 placebo). A treatment effect for NPI-001 Tablets was observed for Ellipsoid Zone (EZ) Area at Month 24 (change from baseline versus Pbo as determined by Spectral Domain Optical Coherence Tomography). In fact, EZ area change from baseline for NPI-001 versus placebo was statistically significantly less at 6, 9, 12, 18 and 24 month timepoints (FIG. 1).

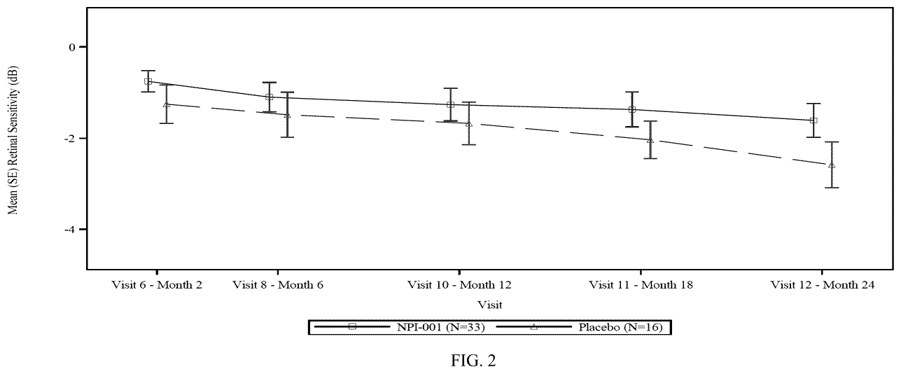

Also, a treatment effect for NPI-001 Tablets was observed for retinal sensitivity by microperimetry (MP) (FIG. 2). Though the effect by MP was not statistically significant at 24 months, the treatment with NPI-001 (blue solid line) appeared to diverge over time from placebo (dashed red line), slowing the retinal sensitivity rate of change over 24 months for NPI-001 by nearly 30% (FIG. 2).

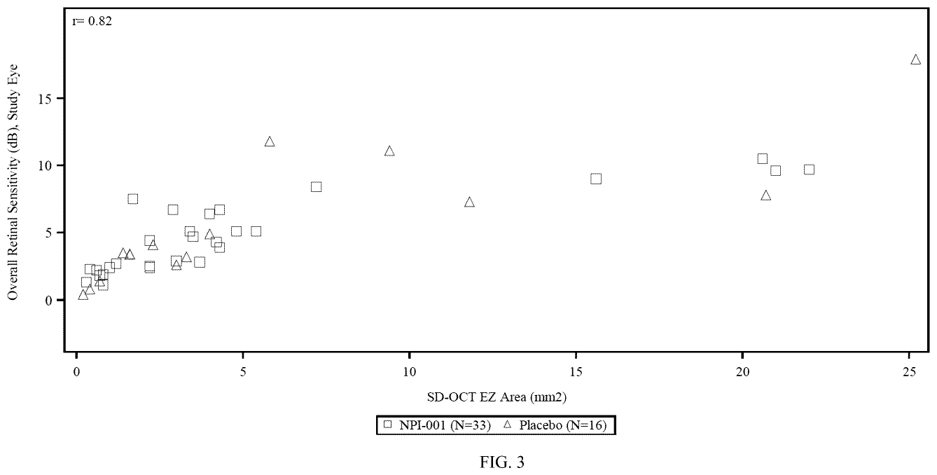

Measurements of EZ area and retinal sensitivity were highly correlated at month 24 (Pearson coefficient) (FIG. 3).

Surprising and unexpected results. These observed treatment effects with NACA (NPI-001) Tablets in patients with UARP are surprising (FIG. 1, FIG. 2, FIG. 3) considering the sample size (49 subjects) of this dataset.

These observed treatment effects with NACA (NPI-001) Tablets in patients with UARP are surprising because such beneficial effects have never been shown by any other drug product on slowing the disease progression in UARP.

These observed effects with NACA (NPI-001) Tablets in patients with UARP are surprising because no other drug study has ever been shown to protect photoreceptor cells in patients with RP or UARP.

The observed effects with NACA (NPI-001) Tablets in patients with UARP for retinal sensitivity and EZ area are surprising (FIG. 1 and FIG. 2) because no other drug study has ever shown a treatment effect in patients with UARP.

The observed effects with NACA (NPI-001) Tablets in patients with UARP are surprising because NPI-001 Tablets, 250 mg, BID, in patients with UARP, cause effects on retinal sensitivity and EZ area that are highly correlated (Pearson coefficient) (FIG. 3).

These observed effects with NPI-001 Tablets in patients with UARP are surprising because no other small molecule drug study has ever shown any kind of treatment effect in patients with RP or UARP.

### LISTING OF EMBODIMENTS

Embodiment 1. A method for treating an eye disease caused by oxidative damage in an animal or human in need thereof, the method comprising: administering to the animal or human an effective amount of an N-acetylcysteine amide (NACA) sufficient to protect vision and inhibit degradation of Ellipsoid Zone (EZ) area and retinal sensitivity.

Embodiment 2. The method of embodiment 1, wherein the eye disease is Usher syndrome associated retinitis pigmentosa (UARP).

Embodiment 3. The method of embodiments 1 or 2, wherein the NACA is provided orally, peritoneally, intravenously, dermally, bucally, sublingually, topically, topical ocularly, intraocularly, intravitreally, transmucosally, or by inhalation.

Embodiment 4. The method of any one of embodiments 1 to 3, wherein the NACA inhibits the reduction in EZ area as measured by Spectral Domain Optical Coherence Tomography (SD-OCT).

Embodiment 5. The method of any one of embodiments 1 to 4, wherein the NACA inhibits the reduction retinal sensitivity as measured by microperimetry.

Embodiment 6. The method of any one of embodiments 1 to 5, wherein the NACA is dosed as one tablet per day or as multiple tablets per day.

Embodiment 7. The method of any one of embodiments 1 to 6, wherein the NACA is dosed greater than 200 mg per day.

Embodiment 8. The method of any one of embodiments 1 to 7, wherein the NACA is dosed at 50, 100, 200, 201, 210, 225, 250, 275, 300, 350, 400, 450, 500, 600, 700, 750, 800, 900, or 1,000 mg per day.

Embodiment 9. The method of any one of embodiments 1 to 8, wherein the NACA is dosed for less than 6 months or for 6, 9, 12, 18, 24, or more than 24 months.

Embodiment 10. A method for treating retinitis pigmentosa associated with Usher syndrome associated retinitis pigmentosa (UARP), the method comprising: identifying that the subject has Usher syndrome associated retinitis pigmentosa (UARP); and administering an effective amount of an N-acetylcysteine amide (NACA) sufficient to inhibit degradation of vision in Usher syndrome associated retinitis pigmentosa (UARP).

Embodiment 11. The method of embodiment 11, wherein the NACA inhibits degradation of Ellipsoid Zone (EZ) area and retinal sensitivity.

Embodiment 12. The method of embodiments 11 or 12, wherein the NACA inhibits a reduction in EZ area as measured by Spectral Domain Optical Coherence Tomography (SD-OCT).

Embodiment 13. The method of any one of embodiments 11 to 13, wherein the NACA inhibits a reduction in retinal sensitivity as measured by microperimetry.

Embodiment 14. The method of any one of embodiments 11 to 14, wherein the NACA is dosed as one tablet per day or as multiple tablets per day.

Embodiment 15. The method of any one of embodiments 11 to 15, wherein the NACA is dosed greater than 200 mg per day.

Embodiment 16. The method of any one of embodiments 11 to 16, wherein the NACA is dosed at 50, 100, 200, 201, 210, 225, 250, 275, 300, 350, 400, 450, 500, 600, 700, 750, 800, 900, or 1,000 mg per day.

Embodiment 17. The method of any one of embodiments 11 to 17, wherein the NACA is dosed for less than 6 months or for 6, 9, 12, 18, 24, or more than 24 months.

Embodiment 18. The method of any one of embodiments 11 to 18, wherein the NACA dose does not reach an appreciable daily steady-state concentration.

Embodiment 19. A method for treating an eye disease caused by oxidative damage in an animal or human in need thereof, the method comprising: identifying that the subject has retinitis pigmentosa associated with Usher syndrome (RP/USH); and administering an effective amount of an N-acetylcysteine amide (NACA) sufficient to inhibit loss of vision.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112 as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Birch D G, Locke K G, Wen Y, Locke K I, Hoffman D R, Hood D C. Spectral-domain optical coherence tomography measures of outer segment layer progression in patients with X-linked retinitis pigmentosa. JAMA Ophthalmol. 2013 September; 131 (9): 1143-50. doi: 10.1001/jamaophthalmol.2013.4160. PMID: 23828615; PMCID: PMC4111937.
Birch D G, Cheng P, Duncan J L, Ayala A R, Maguire M G, Audo I, Cheetham J K, Durham T A, Fahim A T, Ferris F L 3rd, Heon E, Huckfeldt R M, Iannaccone A, Khan N W, Lad E M, Michaelides M, Pennesi M E, Stingl K, Vincent A, Weng C Y; Foundation Fighting Blindness Consortium Investigator Group. The RUSH2A Study: Best-Corrected Visual Acuity, Full-Field Electroretinography Amplitudes, and Full-Field Stimulus Thresholds at Baseline. Transl Vis Sci Technol. 2020 Oct. 8; 9 (11): 9. doi: 10.1167/tvst.9.11.9. PMID: 33133772; PMCID: PMC7552938.
Buckley T M W, Jolly J K, Josan A S, Wood L J, Cehajic-Kapetanovic J, MacLaren R E. Clinical applications of microperimetry in RPGR-related retinitis pigmentosa: a review. Acta Ophthalmol. 2021 December; 99 (8): 819-825. doi: 10.1111/aos.14816. Epub 2021 Mar. 29. PMID: 33783139.
Cross N, van Steen C, Zegaoui Y, Satherley A, Angelillo L. Retinitis Pigmentosa: Burden of Disease and Current Unmet Needs. Clin Ophthalmol. 2022 Jun. 20; 16:1993-2010. doi: 10.2147/OPTH.S365486. PMID: 35757022; PMCID: PMC9232096.
Daiger S P, Sullivan L S, Bowne S J. Genes and mutations causing retinitis pigmentosa. Clin Genet. 2013 August; 84 (2): 132-41. doi: 10.1111/cge.12203. Epub 2013 Jun. 19. PMID: 23701314; PMCID: PMC3856531.
Dong A, Stevens R, Hackett S, Campochiaro P A. Compared with N-acetylcysteine (NAC), N-Acetylcysteine Amide (NACA) Provides Increased Protection of Cone Function in a Model of Retinitis Pigmentosa. Invest Ophthalmol Vis Sci. 2014 Apr. 30; 55 (13): 1736-1736.
Duncan J L, Liang W, Maguire M G, Audo I, Ayala A R, Birch D G, Carroll J, Cheetham J K, Esposti S D, Durham T A, Erker L, Farsiu S, Ferris F L 3rd, Heon E, Hufnagel R B, Iannaccone A, Jaffe G J, Kay C N, Michaelides M, Pennesi M E, Sahel J A; Foundation Fighting Blindness Consortium Investigator Group. Baseline Visual Field Findings in the RUSH2A Study: Associated Factors and Correlation With Other Measures of Disease Severity. Am J Ophthalmol. 2020 November; 219:87-100. doi: 10.1016/j.ajo.2020.05.024. Epub 2020 May 22. PMID: 32446738; PMCID: PMC8596302.
ENCELTO B L A 125798, 2025, accessed 5 Sep. 2025 at: www.fda.gov/vaccines-blood-biologics/center-biologics-evaluation-and-research-cber-product-approval-information/encelto.
Hartel B P, Lofgren M, Huygen P L, et al. A combination of two truncating mutations in USH2A causes more severe and progressive hearing impairment in Usher syndrome type IIa. Hear Res 2016; 339:60-68. Karali M, Testa F, Di Iorio V, Torella A, Zeuli R, Scarpato M, Romano F, Onore M E, Pizzo M, Melillo P, Brunetti-Pierri R, Passerini I, Pelo E, Cremers F P M, Esposito G, Nigro V, Simonelli F, Banfi S. Genetic epidemiology of inherited retinal diseases in a large patient cohort followed at a single center in Italy. Sci Rep. 2022 Dec. 2; 12 (1): 20815. doi: 10.1038/s41598-022-24636-1. PMID: 36460718; PMCID: PMC9718770.
Komeima, K., et al., Antioxidants reduce cone cell death in a model of retinitis pigmentosa. Proc Natl Acad Sci USA, 2006. 103 (30): p. 11300-5.
Komeima K, Rogers B S, Campochiaro P A. Antioxidants slow photoreceptor cell death in mouse models of retinitis pigmentosa. J Cell Physiol. 2007 December; 213 (3): 809-15.
Mehta C R, Pocock S J. Adaptive increase in sample size when interim results are promising: a practical guide with examples. Stat Med. 2011 Dec. 10; 30 (28): 3267-84. doi: 10.1002/sim.4102. Epub 2010 Nov. 30. PMID: 22105690.
Pierrache L H, Hartel B P, van Wijk E, Meester-Smoor M A, Cremers F P, de Bacre E, de Zacytijd J, van Schooneveld M J, Cremers C W, Dagnelie G, Hoyng C B, Bergen A A, Leroy B P, Pennings R J, van den Born L I, Klaver C C. Visual Prognosis in USH2A-Associated Retinitis Pigmentosa Is Worse for Patients with Usher Syndrome Type Ila Than for Those with Nonsyndromic Retinitis Pigmentosa. Ophthalmology. 2016 May; 123 (5): 1151-60. doi: 10.1016/j.ophtha.2016.01.021. Epub 2016 Feb. 27. PMID: 26927203.
Plantinga R F, Pennings R J, Huygen P L, Sankila E M, Tuppurainen K, Kleemola L, Cremers C W, Deutman A F. Visual impairment in Finnish Usher syndrome type III. Acta Ophthalmol Scand. 2006 February; 84 (1): 36-41. doi: 10.1111/j.1600-0420.2005.00507.x. PMID: 16445437.
Schimel A M, Abraham L, Cox D, Sene A, Kraus C, Dace D S, Ercal N, Apte R S. N-acetylcysteine amide (NACA) prevents retinal degeneration by up-regulating reduced glutathione production and reversing lipid peroxidation. Am J Pathol. 2011 May; 178 (5): 2032-43. doi: 10.1016/j.ajpath.2011.01.036. Epub 2011 Mar. 31. PMID: 21457933; PMCID: PMC3081196.
Sengillo J D, Cabral T, Schuerch K, Duong J, Lee W, Boudreault K, Xu Y, Justus S, Sparrow J R, Mahajan V B, Tsang S H. Electroretinography Reveals Difference in Cone Function between Syndromic and Nonsyndromic USH2A Patients. Sci Rep. 2017 Sep. 11; 7 (1): 11170. doi: 10.1038/s41598-017-11679-y. PMID: 28894305; PMCID: PMC5593892.
Zaw K, Carvalho L S, Aung-Htut M T, Fletcher S, Wilton S D, Chen F K, McLenachan S. Pathogenesis and Treatment of Usher Syndrome Type IIA. Asia Pac J Ophthalmol (Phila). 2022 July-August 1; 11 (4): 369-379. doi: 10.1097/APO.0000000000000546. Epub 2022 Aug. 17. PMID: 36041150.

What is claimed is:

1. A method for treating an eye disease caused by oxidative damage in an animal or human in need thereof, the method comprising:
administering to the animal or human an effective amount of an N-acetylcysteine amide (NACA) sufficient to protect vision and inhibit degradation of Ellipsoid Zone (EZ) area and retinal sensitivity.

2. The method of claim 1, wherein the eye disease is Usher syndrome associated retinitis pigmentosa (UARP).

3. The method of claim 1, wherein the NACA is provided orally, peritoneally, intravenously, dermally, bucally, sublingually, topically, topical ocularly, intraocularly, intravitreally, transmucosally, or by inhalation.

4. The method of claim 1, wherein the NACA inhibits the reduction in EZ area as measured by Spectral Domain Optical Coherence Tomography (SD-OCT).

5. The method of claim 1, wherein the NACA inhibits the reduction retinal sensitivity as measured by microperimetry.

6. The method of claim 1, wherein the NACA is dosed as one unit dose (including tablet) per day or as multiple unit doses (including tablets) per day.

7. The method of claim 1, wherein the NACA is dosed at 125 mg per day.

8. The method of claim 1, wherein the NACA is dosed at 125 mg twice, thrice, or four times per day.

9. The method of claim 1, wherein the NACA is dosed greater than 200 mg per day.

10. The method of claim 1, wherein the NACA is dosed at 250 mg once, twice or thrice per day.

11. The method of claim 1, wherein the NACA is dosed at 50, 100, 200, 201, 210, 225, 250, 275, 300, 350, 400, 450, 500, 600, 700, 750, 800, 900, or 1,000 mg per day.

12. The method of claim 1, wherein the NACA is dosed for less than 6 months or for 6, 9, 12, 18, 24, or more than 24 months.

13. A method for treating retinitis pigmentosa associated with Usher syndrome associated retinitis pigmentosa (UARP), the method comprising:
identifying that the subject has Usher syndrome associated retinitis pigmentosa (UARP); and
administering an effective amount of an N-acetylcysteine amide (NACA) sufficient to inhibit degradation of vision in Usher syndrome associated retinitis pigmentosa (UARP).

14. The method of claim 11, wherein the NACA inhibits degradation of Ellipsoid Zone (EZ) area and retinal sensitivity.

15. The method of claim 11, wherein the NACA inhibits a reduction in EZ area as measured by Spectral Domain Optical Coherence Tomography (SD-OCT).

16. The method of claim 11, wherein the NACA inhibits a reduction in retinal sensitivity as measured by microperimetry.

17. The method of claim 11, wherein the NACA is dosed as one tablet per day or as multiple tablets per day.

18. The method of claim 11, wherein the NACA is dosed greater than 200 mg per day.

19. The method of claim 11, wherein the NACA is dosed at 50, 100, 200, 201, 210, 225, 250, 275, 300, 350, 400, 450, 500, 600, 700, 750, 800, 900, or 1,000 mg per day.

20. The method of claim 11, wherein the NACA is dosed for less than 6 months or for 6, 9, 12, 18, 24, or more than 24 months.

21. A method for treating an eye disease caused by oxidative damage in an animal or human in need thereof, the method comprising:
identifying that the subject has retinitis pigmentosa associated with Usher syndrome (RP/USH); and
administering an effective amount of an N-acetylcysteine amide (NACA) sufficient to inhibit loss of vision.
